(19) United States
(12) Patent Application Publication
(10) Pub. No.: US 2024/0379239 A1
(43) Pub. Date: Nov. 14, 2024

(54) AUTOMATED DETECTION AND MANAGEMENT OF VAVLULAR HEART DISEASE USING MACHINE LEARNING

(71) Applicants: GE Precision Healthcare LLC, Wauwatosa, WI (US); Partners HealthCare System, Inc., Boston, MA (US); The General Hospital Corporation, Boston, MA (US); The Brigham and Women’s Hospital, Inc., Boston, MA (US)

(72) Inventors: Eigil Samset, Oppegård (NO); Xiang Li, Chestnut Hill, MA (US); Quanzheng Li, Belmont, MA (US); Michael H. Picard, Carlisle, MA (US); Hui Ren, Boston, MA (US); Carola Alejandra Maraboto Gonzalez, Boston, MA (US); Jerome Charton, Quincy, MA (US); Abhijit Patil, Bengaluru (IN); Mark James Perkins, Knaresborough (GB)

(21) Appl. No.: 18/313,931

(22) Filed: May 8, 2023

(51) Int. Cl. G16H 50/30; G16H 10/60; G16H 50/70

(52) U.S. Cl. CPC G16H 50/30; G16H 10/60; G16H 50/70

(57) ABSTRACT

Techniques are described for computer-implemented techniques for managing various aspects of the cardiac care pathway using machine learning. According to an embodiment, a method can include training an outcomes forecasting model to predict patient outcomes resulting from undergoing a cardiac valve procedure using multi-modal training data for a plurality of different patients, wherein the training comprising separately training different machine learning sub-models of the forecasting model to predict preliminary patient outcome data and mapping the preliminary patient outcome data to the patient outcomes, resulting in a trained version of the outcome forecasting model. The method further includes applying the trained version of the outcomes forecasting model to new multi-modal data for a new patient to predict the patient outcomes for the new patient resulting from undergoing the cardiac value procedure.

### TECHNICAL FIELD

This application relates to computer-implemented techniques for managing various aspects of the cardiac care pathway using machine learning methods, particularly with respect to the early detection and management of patients with valvular heart disease.

### BACKGROUND

The prevalence of valvular heart disease is increasing owing to prolonged life expectancy. Aortic valve stenosis (AS) is the most prevalent of these diseases worldwide and is associated with increased mortality, even when moderate in severity. With no medications proven to attenuate or reverse stenosis progression, the only available treatment is valve replacement, either via surgical aortic valve replacement (SAVR) or via a transcatheter approach, referred to as transcatheter aortic valve replacement (TAVR). These procedures should ideally be performed when the risks of the disease process (e.g., sudden cardiac death, irreversible functional impairment, and heart failure) outweigh those of intervention (e.g., procedural risk, long-term complications, and the potential need for reoperation). However, clinicians frequently lack robust evidence to make accurate assessments of such risk. Therefore, deciding the timing of valvular intervention is difficult in many patients because of the complex interplay of multiple factors that determine the risk.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments described herein, systems, computer-implemented methods, apparatus and/or computer program products are described that provide for managing various aspects of the cardiac care pathway using machine learning, particularly with respect to early detection and management of patients with valvular heart disease.

According to an embodiment, a system is provided that comprises a memory that stores computer-executable components, and a processor that executes the computer-executable components stored in the memory. The computer-executable components can comprise a training component that trains an outcomes forecasting model to predict patient outcomes resulting from undergoing a cardiac valve procedure using multi-modal training data for a plurality of different patients, wherein the training comprising separately training different machine learning sub-models of the forecasting model to predict preliminary patient outcome data and mapping the preliminary patient outcome data to the patient outcomes, resulting in a trained version of the outcome forecasting model. The computer-executable components further comprise an outcomes forecasting component that applies the trained version of the outcomes forecasting model to new multi-modal data for a new patient to predict the patient outcomes for the new patient resulting from undergoing the cardiac value procedure. In one or more embodiments, the patient outcomes can include the length of stay (LOS) and readmission risk. The patient outcomes can also include various other positive and negative clinical and non-clinical outcomes that have been historically observed for different patients that received the cardiac valve procedure.

In some embodiments, the computer-executable components can further comprise a condition assessment component that performs an assessment of valvular heart disease of the new patient based on the analysis of the new multi-modal patient data for the new patient and determines a measure of severity of the valvular heart disease for the new patient based on the assessment, the new muti-modal data representing condition parameters for the patient at different points in time up to a current point in time, and wherein the condition assessment component determines whether the cardiac valve procedure is appropriate for the new patient based on whether the severity measure exceeds a threshold. In some implementations of these embodiments, the outcomes forecasting component applies the trained version of the outcomes forecasting model to the new multi-modal patient data for the new patient based on the severity measure exceeding the threshold.

The computer-executable components can further comprise a data collection component that receives respective components of the new multi-modal patient data via a network from one or more electronic healthcare information sources as the respective components are received at the one or more electronic healthcare information sources, and wherein the condition assessment component performs the assessment repeatedly based on reception of new components of the respective components.

In various embodiments, the cardiac valve procedure comprises different types of procedures and the computer-executable components further comprise a procedure assessment component that determines a preferred procedure type of the different types of procedures for performing on the new patient based on the new multi-modal patient data and the severity measure, and wherein the patient outcomes predicted by the outcomes forecasting model for the new patient are a function of the preferred procedure type. In some embodiments, the surgical cardiac value procedure comprises an aortic valve replacement procedure and wherein the different types of procedures comprise a surgical aortic valve replacement (SAVR) procedure type and a transcatheter aortic valve replacement (TAVR) procedure type. With these embodiments, the procedure assessment component determines the preferred procedure type and one or more replacement valves to use for the procedure based on a plurality of criteria selected from the group consisting of: annulus size, the distance of coronary artery ostia from AV annulus, severe aortic valve calcification, aneurysm of ascending aorta, position and tortuosity of ascending aorta, bicuspid aortic valve, access route tortuosity, small vessel diameter, left ventricular function, coronary artery disease, and ECG conduction abnormalities. Additionally, or alternatively, the cardiac valve procedure may include mitral, tricuspid and pulmonary valve repair and replacement procedures.

The computer-executable component can further comprise a recommendation component that determines a recommendation regarding whether the new patient should proceed or not proceed with the cardiac valve procedure based on the patient outcomes predicted for the new patient and the severity measure, and a reporting component that generates report data identifying the recommendation, the severity measure and the patient outcomes predicted for the new patient and provides the report data to one or more entities using an electronic reporting mechanism. In some embodiments, the recommendation component determines the recommendation based on a value output by a machine learning model generated based on the application of the machine learning model to input data comprising the new multi-modal data, the severity measure, and the patient outcomes predicted for the new patient, the value indicative of the degree to which proceeding with the cardiac valve procedure will improve a quality of life of the patient, wherein the training component trains the machine learning model using a training dataset comprising historical data for past patients including patients that received and did not receive the cardiac valve procedure.

In some embodiments, the computer-executable components further comprise an ordering component that determines one or more medical supplies needed for the proceeding with the cardiac valve procedure based on the recommendation indicating the new patient should proceed with the cardiac valve procedure and orders the one or more medical supplies using an electronic medical supply ordering system.

In some embodiments, elements described in the disclosed systems can be embodied in different forms such as a computer-implemented method, a computer program product, or another form.

### DESCRIPTION OF THE DRAWINGS

FIG. 1 illustrates a block diagram of an example, non-limiting system that facilitates early detection and management of patients with valvular heart disease using machine learning analytics in accordance with one or more embodiments of the disclosed subject matter.

FIG. 2 presents an example server device that facilitates early detection and management of patients with valvular heart disease using machine learning analytics in accordance with one or more embodiments of the disclosed subject matter.

FIG. 3 presents example computer-executable models that facilitate early detection and management of patients with valvular heart disease using machine learning analytics in accordance with one or more embodiments of the disclosed subject matter.

FIG. 4 presents an example graphical user interface (GUI) that facilitates early detection and management of patients with valvular heart disease in accordance with one or more embodiments of the disclosed subject matter.

FIG. 5 illustrates an example ensemble model training process and architecture that facilitates predicting patient outcomes associated with receiving a cardiac valve procedure in accordance with one or more embodiments of the disclosed subject matter.

FIG. 6 presents a flow diagram of an example process for early detection and management of patients with valvular heart disease using machine learning analytics in accordance with one or more embodiments of the disclosed subject matter.

FIG. 7 presents a flow diagram of another example process for early detection and management of patients with valvular heart disease using machine learning analytics in accordance with one or more embodiments of the disclosed subject matter.

FIG. 8 presents an example computer-implemented process that facilitates early detection and management of patients with valvular heart disease in accordance with one or more embodiments of the disclosed subject matter.

FIG. 9 illustrates a block diagram of an example, non-limiting operating environment in which one or more embodiments described herein can be facilitated.

FIG. 10 is a schematic block diagram of a sample-computing environment.

### DETAILED DESCRIPTION

The following detailed description is merely illustrative and is not intended to limit embodiments and/or application or uses of embodiments. Furthermore, there is no intention to be bound by any expressed or implied information presented in the preceding Summary section or in the Detailed Description section.

The disclosed subject matter is directed to systems, computer-implemented methods, apparatus and/or computer program products are described that provide for managing various aspects of the cardiac care pathway using machine learning, particularly with respect to early detection and management of patients with valvular heart disease. The current innovation is part of the cardiac care pathway that spans from precision diagnostics to precision therapeutics to precision monitoring. One specific use case in the care pathway is the early detection of aortic stenosis (AS), and its management. However, the disclosed techniques can also encompass detecting and managing other types of valvular heart disease (e.g., regurgitation, stenosis, prolapse, insufficiency and atresia) affecting any of the heart's four valves (e.g., aortic, mitral, pulmonary and tricuspid valves).

During the patient journey in the cardiac care pathway, once the patient has been diagnosed with a valvular heart disease, such as AS, the question that needs to be answered is when is the appropriate time to intervene, if at all, and perform a cardiac valve treatment procedure, such as SAVR or TAVR. While intervening too early for valve replacement during the longitudinal encounter might expose the patient to risk, such as complication of surgery, living with a prosthetic valve, anticoagulation or repeat intervention for structural valve deterioration, intervening too late might cause an irreversible damage to the myocardium, such as sudden cardiac death, increased peri-operative risk, heart failure, hospital admissions, increased mortality and potential major financial burden to the payer.

According to one or more embodiments, the subject innovation in the care pathway involves staging the valvular heart disease into different severity scores along the longitudinal encounter of the patient using multimodal data available for the patient, such as echocardiogram (ECHO) data, computed tomography (CT) data, electrocardiogram (ECG) data, electronic medical record (EMR) data, clinical reports and notes, patient reported symptoms, medical monitoring device collected data and others. Based on the severity score predicted disease progression and likelihood of a favorable response to therapy, an actionable insight whether to intervene or not is ascertained. One example of intervention in case the predicted positive impact of performing SAVR or TAVR is high is to improve the stroke volume and reduce afterloads by performing a TAVR or SAVR procedure. The innovation involves determining the severity score, predicting disease progression, and estimating the likelihood of a favorable response to therapy, the timing of intervention, and the appropriate procedure to perform on the patient using machine learning techniques based on multitude of criteria such as, but not limited to: annulus size, distance of coronary artery ostia from AV annulus, severe aortic valve calcification, aneurysm of ascending aorta, position and tortuosity of ascending aorta, bicuspid aortic valve, access route (tortuosity of iliac artery/aorta, small vessel diameter, valve-in-vale), medication use, previous procedures performed, hemodynamic parameters, valve morphology, and comorbidities including reduced left- and right ventricular systolic and diastolic function, coronary artery disease, and right- and left bundle branch block.

In order to create a comprehensive care management package, the current innovation trains and applies one or more outcomes forecasting models that forecast one or more outcomes resulting from proceeding with a particular cardiac valve treatment procedure (e.g., TAVR, SAVR, and other types of procedures) for a given patient. In various embodiments, the one or more outcomes forecasting model uses multimodal data (e.g., EMR data, ECHO data, CT data, etc.) for patients diagnosed with a valvular heart disease prior to receiving a treatment procedure to predict various outcomes, including whether the patient will experience a longer than usual length of stay (LOS) after the procedure and whether the patient will be re-admitted to the hospital due to cardiovascular reasons within a defined timeframe post discharge (e.g., one month, three months, six months, etc.). In some embodiments, the LOS and readmission risk predictions can include a more granular output that includes a predicted timeframe (e.g., number of days) of the patient's LOS and/or timing of potential readmission. In some implementations, the input EMR data can be formulated as tabular values, with each row representing a patient and each column representing a specific patient feature, including demographics, laboratories, comorbidities, symptoms, medications and structured echocardiograph reports. In some embodiments, the outputs of the outcomes forecasting model can include two binary values indicating: 1) whether the patient will experience a longer than usual LOS after the procedure and 2) whether the patient will be re-admitted to the hospital due to cardiovascular reasons within six months of discharge. The one or more outcomes forecasting models can also generate other outcomes predictions, including predicted changes to one or more relevant and defined physiological parameters and comorbidities (e.g., left ventricular (LV) function, right ventricular (RV) function, left bundle branch block (LBBB), etc.), predicted changes to the patient's severity measure, and predicted complications or adverse reactions. In some embodiments, some or all predictions generated by the one or more outcomes forecasting models can also include one or more confidence measure that reflect a measure of confidence in the model's prediction output.

In various embodiments, training of the one or more outcomes forecasting models involves ensemble learning of a plurality of different sub-models corresponding to different types of machine learning models. For example, in some implementations, the different types can include, Random Forest (RF), XGBoost, and Non-linear Kernel SVM, which are individually trained for predicting patient outcomes using the multimodal training data as input. In some embodiments, the outcomes forecasting model ensemble is implemented by a two-layer Multilayer Perceptron (MLP), which is trained by mapping the output from the different sub to the patient outcomes. The ensemble outcomes forecasting model demonstrates a much higher prediction performance in the initial validation, compared with commonly used risk score systems, achieving an area under the curve (AUC) that can meet the clinical acceptance criteria set by the physicians.

The techniques described herein include detecting, determining, or otherwise obtaining the large-scale, multi-site, multimodal dataset used to train the severity scoring and outcomes forecasting models. Techniques herein also include designing and implementing ensemble modeling, and the system architecture that provides for automatic input data retrieval and curation from the various disparate electronic data sources/systems via a networked interface. In addition, the disclosed techniques take a holistic approach to model all relevant patient data available from various disparate electronic data/sources and systems as it becomes available over the longitudinal patient journey. In this regard, rather than relying on a limited pre-selected set of patient features related to the valvular heart disease and the treatment procedure, the model collects all the feasible and related EMR data, imaging data, medical monitoring device data and others from the patients and performs a data-driven feature selection to optimize the patient outcome prediction. In some embodiments, by leveraging standardized healthcare information exchange protocols (e.g., Fast Healthcare Interoperability Resources (FHIR) protocols and the like), the disclosed techniques can be run in a fully automated approach, with automatic data retrieval, curation, and prediction occurring repeatedly for the patient over time, thus offering fast model deployment and continuous analysis of the incoming data.

In various embodiments, the disclosed techniques can further provide information regarding valvular heart disease diagnosis, severity, recommended treatment options and predicted patient care outcomes to the patients and/or their care providers in real-time via a suitable rendering application to support the care delivery process. For example, in some implementations, the solution can be integrated as a visualization tile of a care delivery optimization application accessible via a network-based platform (e.g., a web-application, a website, a thin client application), a centralized command center, or another suitable operating environment. The visualization can allow a care provider to view the view the results of the various machine learning models applied to detect and manage patients diagnosed with a valvular heart disease over time. In some implementations, the visualization can specifically identify and/or otherwise call-out patients determined to be severe and/or otherwise recommended to receive a treatment procedure.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

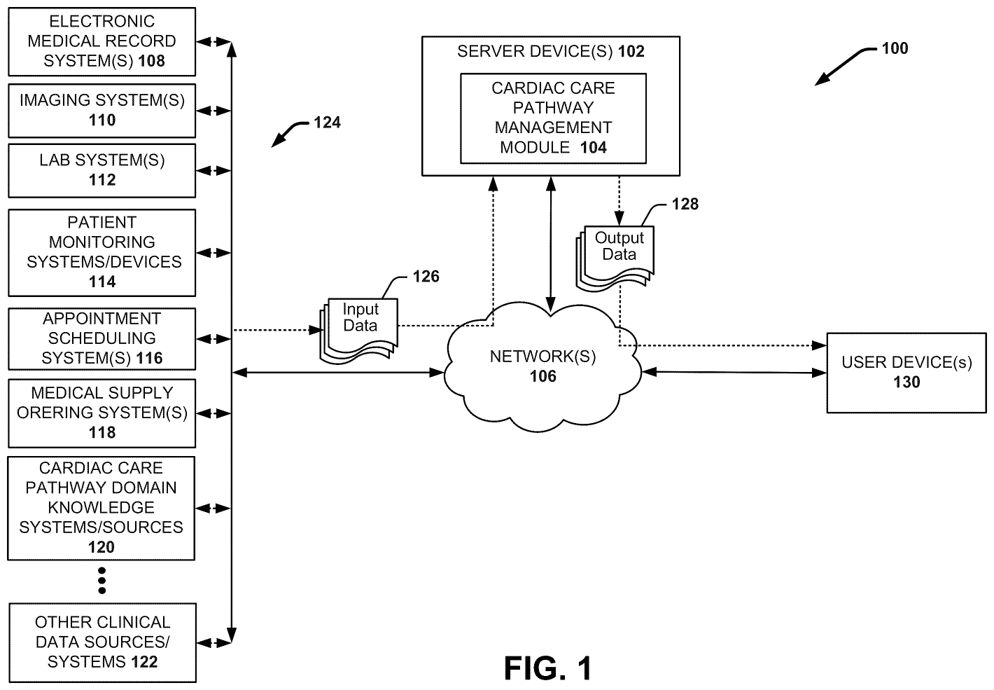

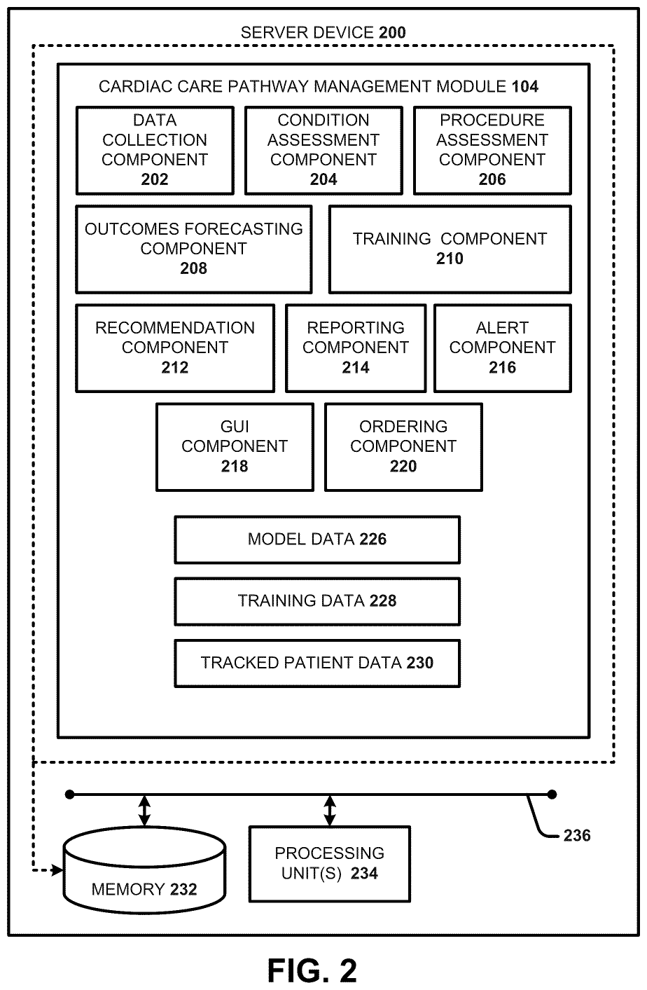

Turning now to the drawings, FIG. 1 illustrates a block diagram of an example, non-limiting system 100 non-limiting system that facilitates early detection and management of patients with valvular heart disease using machine learning analytics in accordance with one or more embodiments of the disclosed subject matter. System 100 includes a plurality of different systems and devices that are communicatively connected to one another via one or more networks 106 which can include any suitable wireless or wired communication network (e.g., the Internet or the like). These systems and devices include one or more server devices 102, electronic healthcare information sources 124, and one or more user devices 130. The one or more server devices 102 that can include a cardiac care pathway management module 104 (and/or components thereof, described with reference to FIG. 2) that can perform a variety of functions related to detecting and managing patients with valvular heart disease. These functions are described in detail with reference to FIGS. 2-8. At a high level, using the cardiac care pathway management module 104, the one or more server devices 102 can collect a variety of input data 126 from a variety of different electronic healthcare information sources 126 pertaining to patients diagnosed (or potentially undiagnosed) with a valvular heart disease. Using the cardiac care pathway management module 104, the one or more server devices 102 can further process the input data 126 to generate output data 128 that facilitates managing the cardiac care pathway of the patients. In various embodiments, the output data 128 can include but is not limited to, information regarding: valvular heart disease diagnosis, disease severity, recommended treatment plan (e.g., one or more recommended procedures to be performed and timing of performance, recommended medical supplies to be utilized for the procedure, and other treatment plan information), alerts (e.g., regarding certain information satisfying an alert criterion, such as highly severe AS diagnosis needing priority treatment), and forecasted outcomes resulting from proceeding with the recommended treatment plan (e.g., forecasted LOS, forecasted readmission rate, forecasted quality of life (QOF) improvement score, forecasted risks, forecasted costs, etc.).

The one or more server devices 102 can further provide (e.g., send, transmit, etc.) the output data 128 to one or more user devices 130 via the one or more networks 106 for rendering at the one or more user devices 130 via any suitable output device (e.g., a display device, a speaker, etc.). For example, the one or more user devices 130 may correspond to devices associated with entities involved in managing and/or treating the patients evaluated by the system 100 (e.g., clinicians, caregivers, friends/family members, etc.), the patients themselves, or another appropriate entity. The one or more user devices can include any suitable computing device capable or communicating information via a network and rendering output data 128 (e.g., mobile phones, smartphones, tablet computer, laptop computer, desktop computers, etc.).

System 100 can be employed to automatically extract and analyze relevant information (i.e., input data 126) for patients from various disparate healthcare information sources 124 as the information becomes populated at the respective healthcare information sources in real-time to provide actionable insights regarding detecting and managing valvular heart disease. For example, the input data 126 (or portions thereof) can correspond to a continuous flow or stream of data for all patients monitored by the system 100 as respective components or portions of the input data 126 is received (e.g., entered, generated, populated, etc.) at the respective healthcare information sources 124 in real-time. As described herein, a real-time computer system can be defined a computer system that performs its functions and responds to external, asynchronous events within a defined, predictable (or deterministic) amount of time. A real-time computer system such as system 100 typically controls a process (e.g., monitoring patient valvular heart disease status and progression, determining treatment needs and timing, forecasting patient outcomes, etc.) by recognizing and responding to discrete events within predictable time intervals, and by processing and storing large amounts of data acquired from the controlled system (e.g., the input data 126). Response time and data throughput requirements can depend on the specific real-time application, data acquisition and critical nature of the type of decision support provided. In the regard, the term “real-time” as used herein with reference to processing and generating information by the cardiac care pathway management module 104 refers to performance of these actions within a defined or predictable amount of time (e.g., a few seconds, less than 10 seconds, less than a minute, etc.) between reception of the input data 126 by the cardiac care pathway management modules 104. Likewise, the term real-time as used with reference to reception of the input data 126 refers to reception of the input data 126 by the cardiac care pathway management module 104 from the respective healthcare information sources 124 within a defined or predictable amount of time (e.g., a few seconds, less than 10 seconds, less than a minute, etc.) after the corresponding information is generated by and/or received by the respective healthcare information sources 124.

The electronic healthcare information sources 124 can be associated with multiple different entities/organizations and/or a single entity/organization. For example, system 100 may be employed by a plurality of different medical care providers (e.g., hospitals, in-patient care facilities, out-patient care facilities, rehabilitation facilities, assisted living facilities, post-acute care facilities, etc.) to continuously and/or regularly track and process relevant data for their patients and received automated insights regarding their valvular heart condition and treatment needs. For example, in some embodiments, one or more features and functionalities of system 100 and/or the cardiac care pathway management module 104 can be provided to healthcare entities as a Platform-as-a-Service (PaaS) and/or a network accessible medical data management system.

Data related to patients generally resides in different electronic healthcare information sources 126 and is generated at various frequencies with respect to time, and/or employs a variety of different technologies. For instance, a vast amount of data is generally generated daily by various network-connected medical devices and/or network-connected medical systems (e.g., medical devices, medical equipment, sensors, mobile devices, controllers, medical logs, etc.) in a healthcare environment. These healthcare information sources can include but are not limited to, electronic medical record systems 108, imaging systems 110, laboratory systems 112, patient monitoring systems/devices 114, appointment scheduling systems 116, Cardiac care pathway domain knowledge systems/sources 120, and other clinical data sources/systems 122. In certain instances, such data can be saved on cloud-based data infrastructure and can be stored as unstructured data. Consequently, searching, retrieving, and/or analyzing the voluminous amounts of unstructured patient data is computationally expensive.

In certain embodiments, the cardiac care pathway management module 104 can employ an application programming interface gateway and/or a microservices architecture to collect and summarize input data 126 received for patients at the respective healthcare information sources 124 over time over their journey. The input data 126 can include patient medical records extracted for patients from one or more electronic medical record (EMR) systems 108. The EMR data can include a variety of rich patient information regarding respective patients' medical history, including family history of heart disease. For example, the EMR data can include information regarding patient current and past reported diagnosis/conditions, past procedures, any implanted medical devices (IMDs), comorbidities (e.g., hypertension, coronary artery disease, atrial fibrillation, left ventricle systolic dysfunction, left ventricle diastolic dysfunction, mitral valve disease, pulmonary hypertension, dyslipidemia, diabetes mellitus, chronic kidney disease and others correlated and potentially correlated to heart disease), patient symptoms (e.g., chest pain, shortness of breath, heat palpitations, fatigue, dizziness, low or high blood pressure, abdominal pain, dyspnea on exertion, syncope, exertional angina, etc.), medications, and patient demographic information (e.g., age, gender, height/weight, body mass index (BMI), ethnicity, occupation, etc.). In some embodiments, the EMR data can also include information regarding behavior activities of patients that can influence heart health, such as but not limited to, diet, exercise activity, stress levels, smoking activity, and alcohol consumption.

The input data 126 can also include medical image data (e.g., medical images and/or imaging studies performed for the respective patients and/or imaging reports providing findings or analysis of the imaging studies (e.g., as performed by a radiologist, a clinician, and/or one or more artificial intelligence models configured to process and analyze the medical image data in association with generating automated findings). The type of imaging data of particular importance with respect to patients diagnosed with or at risk of a valvular heart disease includes image data related to the heart, including echocardiogram (ECO) data (e.g., actual ECO images and/or the corresponding findings report), and chest/heart imaging study data (e.g., actual images and/or the corresponding findings report) captured in all available modalities (e.g., magnetic resonance imaging (MR), conventional X-ray, (XR), computed tomograph (CT) and other medical imaging modalities). The medical image data and/or the corresponding findings reports may be included in the patients EMR and/or extracted from one or more medica imaging systems 108.

The input data 126 can also include electrocardiogram (ECG) data generated for the patients, including raw electrical signals captured via the ECG monitor and/or corresponding findings reports generated based on clinical interpretation of the raw electrical signals (as generated by a clinician/cardiologist and/or an artificial intelligence ECG analysis model). In some embodiments, the ECG data can be included in the patients' EMR data. In other embodiments, the ECG data may be received directly from an ECG monitoring device (e.g., Holter monitor, an implantable loop recorder, or another type of ECG monitoring device) connected to the patient. For example, the patient monitoring devices/systems 114 can include wearable heart monitoring devices that may be worn by patients and configured to record and store and/or report (e.g., via electronic reporting mechanism) ECG data to the one or more server devices 102 at continuous, regular, or event trigger (e.g., activated by the patient in response to detecting systems) times.

The input data 126 can also include other types of physiological data captured and reported via one or more electronic health monitoring devices connected to the respective patients. For example, the patient monitoring systems/devices 114 may include various devices and/or systems that employ various sensors and processing functionality to collect, determine and track information regarding various physiological measurements of the patients in real-time and/or at regular intervals (e.g., hourly, daily, weekly, etc.), including but not limited to, vital signs (e.g., heart rate, blood pressure, blood glucose levels, oxygen saturation, temperature, respiratory rate, etc.), sleep patterns, activity levels, energy levels, fatigue levels, stress levels, weight the current location of the patients, the events and conditions that occur throughout the patient's journey, the clinical status of the patient's, the physiological status of the patients (e.g., vitals) and so on. With these embodiments, the patient monitoring systems/devices 114 can further be configured to provide (e.g., send, transmit, etc.) the physiological measurements to the one or more server devices 102 in real-time, according to a schedule (e.g., hourly, daily, etc.), and/or in response to a defined trigger event (e.g., a measurement exceeding a threshold, a measurement change exceeding a threshold, or satisfying another defined trigger criterion).

The input data 126 can also include laboratory data (e.g., laboratory reports) generated for the patients. For example, the patient flow data 110 can include information reported by one or more laboratory system 114 identifying lab orders, lab results and so one, reported in real-time. In some embodiments, the laboratory data can include results of other forms of tests used to diagnose heart valve disease, including those currently developed (e.g., cardiac catheterization results) and developed in the future.

The electronic medical information sources 126 can also include one or more appointment scheduling systems 116, one or more medical supply ordering systems, 110, cardiac care pathway domain knowledge sources/systems 120 and other clinical data sources/systems. In some embodiments, the cardiac care pathway management module 104 can interface with one or more appointment scheduling systems 116 to automatically schedule patient appointments in accordance with recommended treatment plans. The cardiac care pathway management module 104 can interface with one or more medical supply ordering systems 110 to automatically order one or more medical supplies (e.g., artificial valves, stents, etc.) needed for recommended treatment procedures. The cardiac care pathway management module 104 can employ the cardiac care pathway domain knowledge systems/sources to facilitate generating inferences and/or determination regarding valvular heart disease diagnosis, severity, treatment planning and outcome forecasting.

FIG. 2 presents an example server device 200 that facilitates early detection and management of patients with valvular heart disease using machine learning analytics in accordance with one or more embodiments of the disclosed subject matter. With reference to FIGS. 1 and 2, in various embodiments, the one or more server devices 102 can corresponds to server device 200 or vice versa. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

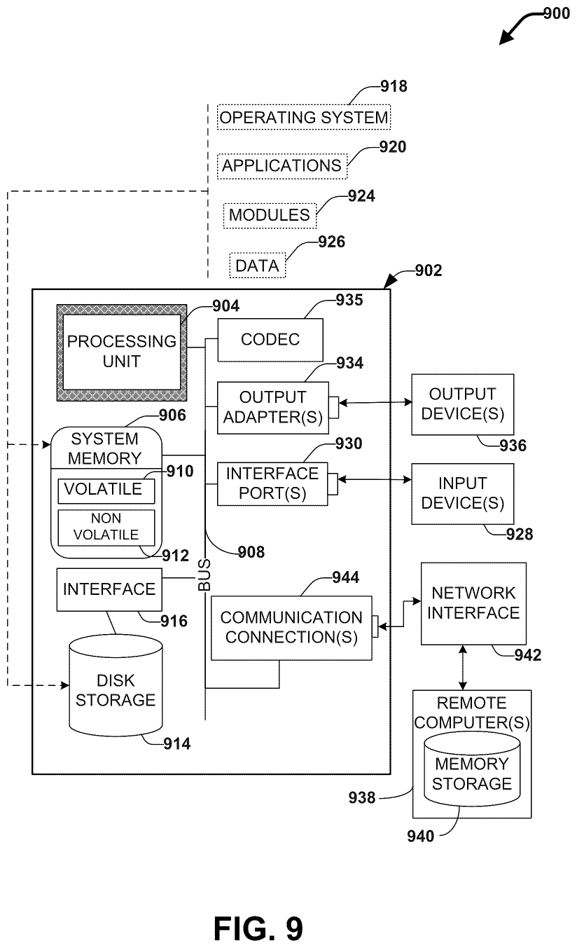

In this regard, the cardiac care pathway management module 104 can correspond to a computer-executable component and includes several computer-executable components configured to perform various different functions associated with detecting and managing valvular heart disease patients. These components include, but are not limited to, data collection component 202, condition assessment component 204, procedure assessment component 206, outcomes forecasting component 208, training component 210, recommendation component 212, reporting component 214, alert component 216, graphical user interface (GUI) component 210, and ordering component 220. These computer/machine executable components can be stored in memory associated with the one or more machines. The memory can further be operatively coupled to one or more processing units such that the components can be executed by the one or more processing units to perform the operations described. For example, in some embodiments, these computer/machine executable components can be stored in memory 232 of the computing server device 200 which can be coupled to one or more processing units 234 for execution thereof. Examples of said and memory and processing units as well as other suitable computer or computing-based elements, can be found with reference to FIG. 9, and can be used in connection with implementing one or more of the systems or components shown and described in connection with FIG. 1 or other figures disclosed herein). The sever device 200 can further include a device bus 236 that couples the memory 232 and the processing unit 124 to one another.

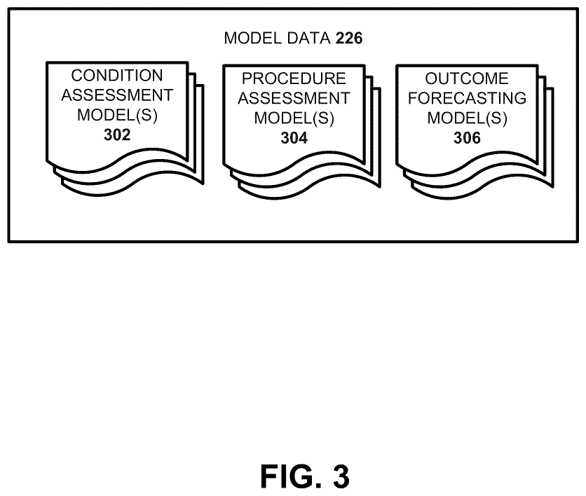

Memory 232 can further store a variety of information that is received by, used by, and/or generated by the server device 200 in association with monitoring and evaluating patient data with respect to detecting and managing patients with valvular heart disease. In the embodiment shown, this information includes (but is not limited to), model data 226, training data 228 and tracked patient data 230. The model data 226 can include a plurality of different models or algorithms that are utilized by the cardiac care pathway management module 104 to generate the output data 128, as illustrated in FIG. 3. With reference to FIGS. 1-3, in various embodiments, the model data 226 can include one or more condition assessment models 302, one or more procedure assessment models 304, one or more outcomes forecasting models 306. These algorithms or models can include several different machine learning or artificial intelligence models and/or heuristic models that are adapted to generate one or more portions of the output data 128 and/or information used to determine or predict one or more portions of the output data 128. In various embodiments, some or all of these models can be trained and regularly updated/refined (e.g., retrained) by the cardiac care pathway management module 104 (via training component 210) using training data 228 and/or the tracked patient data 230, as described in greater detail infra. The tracked patient data 230 can include indexed patient information collected and determined for respective patients evaluated/monitored by the system 100 over time. It should be appreciated that the model training process employed by training component 210 to train one or more of the conditions assessment models 302, the procedure assessment models 304 and/or the outcome forecasting models 306 can vary based on the type machine learning algorithms employed for the respective models, which can vary. For example, some suitable machine learning algorithms/models that can be used for some or all of the models can include but are not limited to: a nearest neighbor algorithm, a naïve Bayes algorithm, a decision tree algorithm, a boosting algorithm, a gradient boosting algorithm, a linear regression algorithm, a neural network algorithm, a k-means clustering algorithm, an association rules algorithm, a q-learning algorithm, a temporal difference algorithm, a deep adversarial network algorithm, a neural network model, a deep learning neural network model, or a combination thereof.

The data collection component 202 can collect (e.g., receive, extract, etc.) the input data 126 for respective patients evaluated/monitored by the system 100 from the one or more electronic healthcare information sources 124 via the one or more networks 106. In some embodiments, all or portions of the input data 126 collected for respective patients can be indexed as a function of respective patient identifiers and time in memory 232 as tracked patient data 230. In some embodiments, the specific patients for which the input data 126 pertains can include predefined patients. The predefined patients may correspond to patients that have received a diagnosis of a valvular heart disease (e.g., AS or another valvular heart disease) that may be suitable candidates for a treatment procedure, e.g., TAVR (transcatheter AV replacement), SAVR (Surgical AV replacement), or another cardiac valvular heart procedure). The predefined patients can also include patients that have received a valvular heart procedure. In this regard, system 100 can be adapted to monitor and evaluate patients post procedure in association with evaluating their care outcomes and updating one or more machine learning models utilized by the cardiac care pathway management module 104 (e.g., used to diagnose, classify severity, determine treatment needs and timing, and forecast care outcomes) and in association with a continuous machine learning regime. The predefined patients may also include patients that have been flagged by a clinician as having a potential to develop a valvular heart disease. Still in other embodiments, the predefined patients may include any predefined group of patients having any clinical diagnosis (e.g., all patients under the current care of a healthcare organization, a select group of patients grouped by diagnosis, comorbidities, age, etc.). For example, system 100 may be applied to regularly evaluate relevant patient data received for all patients to detect patients that may be currently undiagnosed yet exhibit signs and symptoms of a valvular heart disease.

As described above with reference to system 100, in various embodiments, the input data 126 can correspond to a continuous, real-time stream of patient data collected for respective patients as respective components of the input data 126 are generated or received at the respective electronic healthcare information sources 126. For example, in some embodiments, in association with performing an initial evaluation of a patient, the data collection component 202 can extract the entirety of the patient's existing EMR data, imaging data, laboratory data, etc. where the data is located, which can include disparate sources/systems. The data collection component 202 can thereafter extract any updates to the patient's data as they are received/entered at the respective electronic healthcare information sources 124 in association with performing continued analysis of the patient over time. In other words, the data collection component 202 can collect and track any newly received/generated data for the patient over time as it becomes available (e.g., generated, received by, populated within, etc.) at the respective electronic healthcare information sources 124. For example, the data collection component 202 can regularly and/or continuously monitor the respective healthcare information sources 124 to identify any new patient data available for the patient and collect the new patient data as is becomes available. In some embodiments in which the patient utilizes one or more patient monitoring systems/devices 114 to generate physiological data (e.g., ECG data, heart rate, respiratory rate, blood sugar, stress levels, fatigue levels, etc.), the data collection component 202 can receive the physiological data from the monitoring device as it is generated in real-time and/or according to another continuous or regular schedule (e.g., hourly, daily, in response to a trigger event, etc.).

In this regard, the course of care for patients that have been diagnosed with or potentially diagnosed with a vascular heart disease generally involves regular patient encounters involving clinical evaluations preformed by one or more clinicians (e.g., doctor, cardiologist, screening/detection procedure clinician, etc.) over time. During respective patient encounters, the current condition of the patient is evaluated to ascertain symptoms, perform diagnostic tests/procedures (e.g., ECHOs, EGG, stress test, etc.) and relevant measurements/parameters are determined and recorded in the patients' EMR and/or other electronic healthcare information sources (e.g., imaging systems 110, laboratory systems 112, etc.) in the form of evaluation reports, diagnostic reports, physician notes, clinical orders etc. To this end, the data collection component 202 can collect all patient information generated for each patient encounter over time and track the information as a function of time (e.g., in the tracked patient data 230) to generate a longitudinal record of all relevant information for the patient up to the current point in time.

For each patient monitored/tracked by the system 100 (e.g., each patient for which input data 126 is collected/received), the condition assessment component 204 performs a valvular heart disease status assessment of the patient based on the analysis of all multi-modal data collected/received for the patient up to the current point in time. In some embodiments, the valvular heart disease status assessment can involve determining or inferring a current valvular heart disease diagnosis of the patient, including determining or inferring whether the patient has a valvular heart disease, determining or inferring the type of the valvular heart disease (e.g., AS or another type of valvular heart disease), determining or inferring a stage of the valvular heart disease, and determining or inferring a measure of severity of the valvular heart disease. In various embodiments, one or more of these parameters may be included/provided in the patient data collected for the patient (e.g., as entered into the patient's EMR based on clinical evaluation and/or as included in one or more diagnostic reports as ascertained by a clinician and/or an artificial intelligence model based on interpretation of Echo study data, ECG data, imaging data, etc.). In some implementations of these embodiments, the condition assessment component 204 can identify and extract these parameter values directly from the collected patient data. Additionally, or alternatively, the condition assessment component 204 can perform an independent assessment of the patients condition to determine or infer one or more of these parameter values using machine learning and artificial intelligence analytics based collected multi-modal data for the patients representing physiological condition parameters for the patient at different points in time up to a current point in time.

In this regard, in some embodiments, the condition assessment component 204 can apply one or more condition assessment models (e.g., condition assessment models 302) included in the model data 226 to the multi-modal patient data received for the patient to determine or infer whether the patient has a valvular heart disease, determining or inferring the type of the valvular heart disease (e.g., AS or another type of valvular heart disease), determining or inferring a stage of the valvular heart disease, and determining or inferring a measure of severity of the valvular heart disease. The one or more condition assessment models can include one or more machine learning models that have been trained to generate one or more of these parameters based on learned correlations and patters between a plurality of input features extracted from the multi-modal patient data. In some embodiments, these one or more machine learning models include models that have been trained by the cardiac care pathway management module 104 using the training component 210.

In some embodiments, the one or more condition assessment models 302 can include a severity scoring model that generates a severity score measure for a patient that represents a measure of severity of a valvular heart disease of the patient. The severity scoring model can account for a plurality of different features extracted from the multimodal patient data, including but not limited to: ECO study finding, ECG study findings, imaging study findings, patient symptoms (e.g., dyspnea on exertion, syncope, exertional angina, etc.), physiological parameters, comorbidities (e.g., hypertension, coronary artery disease, atrial fibrillation, left ventricle systolic dysfunction, left ventricle diastolic dysfunction, mitral valve disease, pulmonary hypertension, behavioral activities (e.g., smoking activity, alcohol consumption, exercise activity, stress levels, fatigue levels, etc.), medications, and demographic features (e.g., age, gender, ethnicity, BMI, etc.). In various embodiments, in addition to the above-noted features, as applied to AS patients, the severity scoring model can process some or all of the following input features as extracted from the multi-modal patient data to generate the measure of severity of the aortic valve stenosis disease of the patient: annulus size, distance of coronary artery ostia from AV annulus, degree of severe aortic valve calcification, degree of aneurysm of ascending aorta, position and tortuosity of ascending aorta, bicuspid aortic valve characterizes, access route characteristics (e.g., tortuosity of iliac artery/aorta, small vessel diameter, valve-in-valve ratio), degree of left and right ventricular dysfunction, degree of coronary artery disease, and ECG condition abnormalities.

The severity scoring model can further account for previously determined severity scores generated for the patient in the past and up to the current point in time. In this regard, the severity scoring model can generate a severity score for the patient that is based on the progression (e.g., amount and rate) of severity of the disease over time. It should be appreciated that the particular set of input features available each time the condition assessment component 202 determines or infers the severity measure for a patient will change over time over the longitudinal care of the patient. In this regard, the condition assessment component 302 can regularly determine an updated severity scores for the patients as new input data is received and/or on regular basis (e.g., according to a defined schedule, such as daily, weekly, etc.) to facilitate catching changes in the severity of their condition as soon as they arise. It should be appreciated that the amount of patient data processed for each patient evaluated by the system as aggregated over time multiplied by the number of patients monitored by the system 100 (e.g., hundreds, thousands, hundreds of thousands, or more) to generate a severity measure for each patient at this rate would be essentially impossible for a human mind to accomplish. Accordingly, by employing artificial intelligence and machine learning analytics to assess the patient's condition and severity on a continuous basis as new input data is received, the disclosed techniques provide a sustainably technical benefit for patients and their clinical care providers with respect to early detection of vascular heart disease of varying degrees of severity utilizing highly technical means.

For each patient monitored/tracked by the system 100 (e.g., each patient for which input data 126 is collected/received), the procedure assessment component 206 can further perform a procedure assessment for the patient based on the analysis of all multi-modal data collected/received for the patient up to the current point in time, including outputs determined by the condition assessment component 204 (e.g., vascular heart disease diagnosis, disease type, and severity measure or score). The procedure assessment can involve determining or inferring whether an intervention procedure (e.g., SAVR, TAVR or another type of procedure is recommended for the patient, determining or inferring the preferred or optimal treatment procedure for the patient, determining or inferring the timing of need of the procedure (e.g., urgent or as soon as possible, within the next week, within the next month, within the next 6 months, etc.), and determining or inferring the one or more suitable and/or preferred replacement valves suitable for the patient. To facilitate this end, the procedure assessment component 204 can employ one or more procedure assessment models 304 included in the model data 226.

The one or more procedure assessment models 304 can include machine learning models and/or statistical models configured to generate one or more of the above noted outputs (e.g., a value indicative or measure indicative of a degree to which a particular cardiac valve procedure is appropriate for the patient, a timing of need of the procedure and/or one or more suitable and/or preferred replacement valves suitable for the patient) based on a plurality of input features extracted from the multi-modal patient data collected and tracked for the patient over time. These input features can include but are not limited to: disease type diagnosis, disease severity (e.g., the severity measure determined by the condition assessment component 202), ECO study finding, ECG study findings, imaging study findings, patient symptoms (e.g., dyspnea on exertion, syncope, exertional angina, etc.), physiological parameters, comorbidities (e.g., hypertension, coronary artery disease, atrial fibrillation, left ventricle systolic dysfunction, left ventricle diastolic dysfunction, mitral valve disease, pulmonary hypertension, behavioral activities (e.g., smoking activity, alcohol consumption, exercise activity, stress levels, fatigue levels, etc.), medications, and demographic features (e.g., age, gender, ethnicity, BMI, etc.). In various embodiments, in addition to the above noted features, as applied to AS patients, the input features can include some or all of the following features depending on availability for the patient: annulus size, distance of coronary artery ostia from AV annulus, degree of severe aortic valve calcification, degree of aneurysm of ascending aorta, position and tortuosity of ascending aorta, bicuspid aortic valve characterizes, access route characteristics (e.g., tortuosity of iliac artery/aorta, small vessel diameter, valve-in-valve ratio), degree of left and right ventricular dysfunction, degree of coronary artery disease, and ECG conduction abnormalities. In some embodiments, the one or more procedure assessment models 304 can include one or more decision trees that are invoked by the procedure assessment component to determine one or more preferred procedure types for the patient, rank the preferred procedure types in terms of most preferred to least preferred, determine the one or more preferred replacement valves for the procedure, and/or rank the replacement valves in terms of preference priority.

In some embodiments, the one or more procedure assessment models 304 can include machine learning models that are trained by the training component 210 using training data 228. In some embodiments, the one or more procedure assessment models can be tailored to account for a specific procedure type of a plurality of different procedure types (e.g., SAVR, TAVR and other cardiac valve procedure types, including but not limited to, mitral, tricuspid and pulmonary valve repair and replacement procedures). For example, in one embodiment, the procedure assessment models 304 can include a SAVR model configured to generate output data providing a measure of appropriateness of performing the SAVS procedure on the patient, a recommended timeframe for performing the procedure within, and/or one or more recommended replacement valves to use for the patient for the procedure. The one or more procedure assessment models 304 can also include a TAVR model configured to generate output data providing a measure of appropriateness of performing the TAVR procedure on the patient, a recommended timeframe for performing the procedure within, and/or one or more recommended replacement valves to use for the patient for the procedure. With these embodiments, the procedure assessment component 206 can apply both the SAVR model and the TAVR model to the patient data to generate the corresponding output data, and the recommendation component 212 can recommend either the particular procedure best suited for the patient based on the procedure with the higher or more favorable appropriateness score.

In some embodiments, the procedure assessment component 206 can perform the procedure assessment based on the severity measure/score exceeding a predefined threshold (e.g., the procedure assessment may only be performed if the patient has a severity score above the predefined threshold). In other embodiments, the procedure assessment component can perform the procedure assessment regardless of the severity score value and/or based on the severity score value exceeding a low bar threshold. For example, in some cases, patients diagnosed with low or moderate AS may benefit from receiving TAVR and/or SAVR depending on a complex interplay of various factors (e.g., comorbidities, age, medications, etc.).

For each patient monitored/tracked by the system 100 and/or for subset of the patients satisfying one or more defined criterion (e.g., having a severity score exceeding a threshold, having procedure appropriateness score exceeding another threshold, etc.) the outcomes forecasting component 208 can forecast or predict patient outcomes resulting from undergoing one or more cardiac value procedures. To facilitate this end, the outcomes forecasting component 208 can apply one or more outcomes forecasting models 306 included in the model data 226 to a plurality of input features extracted from the multi-modal patient data collected and tracked for the patient over time. In some embodiments, the input to the outcomes forecasting models 308 can also include the outputs determined or inferred by the condition assessment component 204 and the procedure assessment component. For example, the one or more outcomes forecasting models 306 can predict one or more patient outcomes based on the valvular heart disease diagnosis (e.g., disease type), severity measure/score, measure of appropriateness of the one or more cardiac valve procedures, recommended time window for performance of the procedures and/or recommended replacement valve types to be used for the procedures. Additionally, or alternatively, the one or more outcomes forecasting models can process input available input features for the patient selected from the group consisting of: disease type diagnosis, disease severity (e.g., the severity measure determined by the condition assessment component 202), ECHO study finding, ECG study findings, imaging study findings, patient symptoms (e.g., dyspnea on exertion, syncope, exertional angina, etc.), physiological parameters, comorbidities (e.g., hypertension, coronary artery disease, atrial fibrillation, left ventricle systolic dysfunction, left ventricle diastolic dysfunction, mitral valve disease, pulmonary hypertension, behavioral activities (e.g., smoking activity, alcohol consumption, exercise activity, stress levels, fatigue levels, etc.), medications, demographic features (e.g., age, gender, ethnicity, BMI, etc.), annulus size, distance of coronary artery ostia from AV annulus, degree of severe aortic valve calcification, degree of aneurysm of ascending aorta, position and tortuosity of ascending aorta, bicuspid aortic valve characterizes, access route characteristics (e.g., tortuosity of iliac artery/aorta, small vessel diameter, valve-in-valve ratio), degree of left and right ventricular dysfunction, degree of coronary artery disease, and ECG conduction abnormalities.

In some embodiments, the one or more outcomes forecasting models can include separate models tailored to different procedure types of a plurality of different procedure types. For example, the one more outcomes forecasting models 306 can include one or more first models configured to predict patient outcomes resulting from undergoing a TAVR procedure and one second models configured to predict patient outcomes resulting from undergoing a SAVR procedure. Additionally, or alternatively, different procedure types can be accounted for within the outcomes forecasting model, wherein the recommended or most appropriate procedure determined for the patient can be applied as input to the outcomes forecasting model.

In various embodiments, the patient outcomes predicted by the one or more outcomes forecasting models 304 can include LOS and readmission rate. For example, the LOS measure can include a predicted LOS of the patient in the hospital or another inpatient facility following performance of the cardiac valve procedure (e.g., TAVR and/or SAVR or another procedure). The readmission rate can include a predicted probability of readmission of the patient to a hospital or similar inpatient facility following discharge within a defined timeframe after performance of the procedure. In some embodiments, the defined timeframe is six months, however the models can be tailored to predict one or more readmission timeframe probabilities (e.g., one month, three months, six months, one year, etc.). In some embodiments, the LOS and readmission risk predictions can include a more granular output that includes a predicted timeframe (e.g., number of days) of the patient's LOS and/or timing of potential readmission.

Additionally, or alternatively, the patient outcomes predicted by the one or more outcomes forecasting models can include other potential outcomes an their probability of occurrence that may result from undergoing one or more treatment procedures (e.g., TAVR and/or SAVR or another procedure), including but not limited to, one or more adverse outcomes or complications that may arise (e.g., complications of surgery, risk of heart failure, etc.), and other clinical and financial risks that have been correlated to the respective procedures and the respective patient profiles (e.g., wherein the respective patient profiles can account for a multitude of different clinical and demographic attributes associated with the respective patients). The one or more outcomes forecasting models can also generate other outcomes predictions, including predicted changes to one or more defined relevant physiological parameters and comorbidities (e.g., LV/RV function, LBBB, etc.), predicted changes to the patient's severity measure, and others. In some embodiments, some or all predictions generated by the one or more outcomes forecasting models can also include one or more confidence measures that reflect a measure of confidence in the model's prediction output.

In some embodiments, the one or more outcomes forecasting models can include one or more quality of life (QOF) forecasting models adapted to forecast one or more QOL measures indicative of a degree to which the patient's quality of life will be improved as a result of proceeding with one or more cardiac valve treatment procedures. The one or more QOF models can account for different procedure types of a plurality of different procedure types (e.g., TAVR, SAVR and/or other procedure types), and provide a QOF measure tailored as a function of procedure type. The QOF measure can provide a holistic valuation of whether and to what extent the patients quality of life will improve as a result of the procedure, accounting for a plurality of different factors that influence quality of life. In various embodiments, the one or more QOF models can apply some or all of the previous inputs features described above, along with the other forecasted patient outcomes (e.g., LOS, readmission rate, risk of complications, etc.) determined or inferred by the outcomes forecasting component 208. The one or more QOF models can also account for expected financial costs to the patient as result of proceeding with the procedure. In one or more embodiments, wherein the training component 210 trains the one or more QOF models using a training dataset (e.g., included in training data 228) comprising historical data for past patients including patients that received and did not receive the cardiac valve procedure. The training data can include one or more subjective and/or objective measures for the past patient that indicate a measure of their quality of life improvement or deterioration as result of receiving the treatment procedure or forgoing the procedure after one or more periods of time post reception of the procedure and post diagnosis.

The recommendation component 212 can determine a recommendation regarding whether a patient evaluated by the system 100 patient should proceed or not proceed with one or more cardiac valve procedures based on the outputs of the condition assessment component 204, the procedure assessment component 206 and/or the outcomes forecasting component 212. In some embodiments, the recommendation component 204 can determine whether a patient should receive or a TAVR or SAVR procedure based on one or more of the following: their disease severity score, appropriateness scores for the respective procedures, and expected outcomes values (e.g., LOS, readmission risk, risks of other complications and QOS improvement measure). In some embodiments, the recommendation component 212 can recommend a patient proceed with a TAVR or SAVR procedure based on a cost benefit analysis that factors and weights all of the outputs of the condition assessment 204, the procedure assessment component 206 and the outcomes forecasting component 208.

The reporting component 214 can further generate reporting data comprising some or all of the outputs determined or inferred by the condition assessment component 204, the procedure assessment component 206 the outcomes forecasting component 208 and the recommendation component 212. For example, in various embodiments, the report data reported by the reporting component 214 can correspond to the output data 128 or components thereof. The reporting component 214 can further provide the report data to one or more entities using one or more electronic reporting mechanisms. For example, in some embodiments, the reporting component 214 can provide the report data to one or more entities (e.g., the patient and/or the patient's care provider/team) via an electronic message (e.g., an email, a text message, etc.) sent/transmitted via any suitable electronic messaging system or application. In some embodiments, the reporting component 214 can send or provide the electronic message to respective user devices 130 of the entities. In other embodiments, the reporting component 214 can be configured to provide the report data directly to one or more of the electronic healthcare information sources 124. For example, in some embodiments, the reporting component 214 can provide the report data to the patient's EMR, an imaging system 110 or device (e.g., an ECG machine from which ECG results are received) a patient monitoring system/device 114 or the like). In some embodiments, the electronic message may include a link or prompt to access and review a more granular report (e.g., via a dashboard or the like) having additional details of some or all of the outputs determined or inferred by the condition assessment component 204, the procedure assessment component 206 the outcomes forecasting component 208 and the recommendation component 212.

In some embodiments, the alert component 216 can generate and provide alerts to one or more entities regarding one or more of the outputs generated by the condition assessment component 204, the procedure assessment component 206, the outcome forecasting component 208 and/or the recommendation component 212 satisfying one or more alert criteria. For example, in some implementations, an alert criterion can include a change in valvular disease diagnosis or severity for a patient (e.g., as determined based on the output of the condition assessment component 204 relative to a previously determined value determined and tracked for the patient in the tracked patient data 230). In this regard, all outputs generated by the condition assessment component 204, the procedure assessment component 206, the outcomes forecasting component and the recommendation component 212 for each patient over time can be logged and tracked with their patient data in the tracked patient data 230. In another example, an alert criterion can include a disease severity measure/score exceeding a threshold severity level. In another example, an alert criterion can include a determination (e.g., determined or inferred by the procedure assessment component 206) that a patient should receive a TAVR or SAVR procedure, or a determination that the patient should receive the procedure within a timeframe classified as urgent (e.g., within the next 24 hours, within the next week, within the next month, etc.). In another example, an alert criterion can include a forecasted outcome generated by the outcomes forecasting component satisfying an alert criterion, such as a predicted LOS exceeding a threshold LOS (e.g., a LOS exceeding a duration considered normal) and/or a readmission risk exceeding a threshold readmission risk (e.g., a readmission risk considered higher than acceptable withing a specified time window, such as six months following discharge post procedure or another defined time window).

In some embodiments, the report data generated by the reporting component 214 and/or the alerts generated by the alert component 216 can be provided to one or more users (e.g., via their respective user devices 130) via a graphical user interface generated by the GUI component 210. With these embodiments, the GUI component 210 can generate a GUI that can be displayed at the respective user devices using any suitable network accessible platform (e.g., a website, a web application, a mobile application, a thin client application, a thick client application, etc.). The GUI can provide a summary of some or all of the data determined or inferred by the condition assessment component 204, the procedure assessment component 206, the outcomes forecasting component 208 and/or the recommendation component 212 for respective patients evaluated by the system 100. In some implementations in which alerts are generated for a patient, the GUI can include information (e.g., visual indicators and/or audible alarms) that calls out or otherwise highlights the alerts so as to call attention to the alerts.

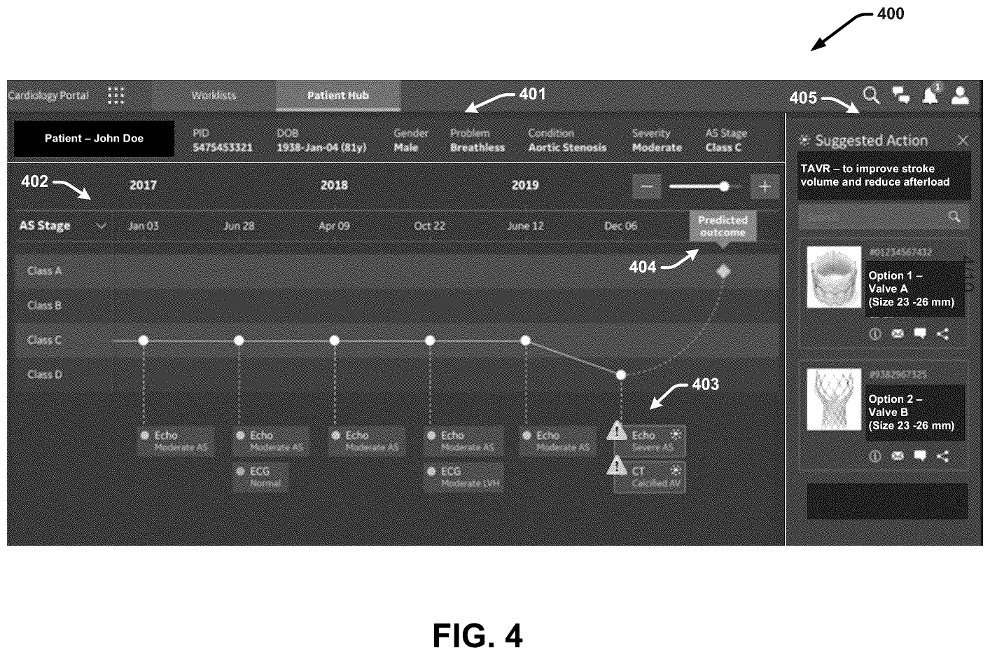

FIG. 4 presents an example GUI 400 that can be generated by the GUI component 210 and presented to users (e.g., clinicians, patients, etc.) providing a summary of relevant information received and/or determined for a patient monitored by system 100 in accordance with various embodiments disclosed herein. The example GUI 400 includes an upper information panel providing information regarding the patient to which the data pertains, including patient identifying information (e.g., name, identification number, date of birth, and gender), a primary reported problem currently being experienced by the patient (e.g., breathlessness), the condition diagnosis (e.g., aortic stenosis), the current severity measure (e.g., moderate) and the current AS severity class stage (e.g., class C). The GUI 400 further provides timeline summary 402 of respective patient encounters as a function of time, diagnostic results generated/determined for the respective encounters, which in this example include Echo findings, ECG findings and CT imaging study findings, and the determined AS stage classification at each encounter. The GUI further calls out the most recent diagnostic findings 403 with alert symbols to indicate the most recent findings satisfy an alert criterion, which in this example is based on the most recent Echo results indicating the AS condition has intensified to a class D with an Echo examine indicating the patient has become severe (e.g., received a severe AS classification) and CT findings indicating the patient's aortic valve has become calcified. The GUI further includes a predicted outcome icon 404 that indicates the cardiac care pathway management module has performed a procedure assessment, a forecasted outcomes assessment, and generated a recommended or suggested action for the patient (e.g., using procedure assessment component 206 and outcomes forecasting component 208, and recommendation component 212). In accordance with this example, the predicted outcome icon 404 can be selected to present the user with the predicted outcomes as a result of proceeding with the suggested action, which in this case is a to perform a TAVR procedure to improve stroke volume and reduce afterload, as indicated in the suggested action section 405. The suggested action section further identifies recommended valve options to use for the procedure including the recommended size (e.g., as determined by the procedure assessment component 206).

With reference again to FIGS. 1-3, in some embodiments in which the cardiac care pathway management module 104 determines or infers (e.g., via procedure assessment component 206 and/or recommendation component 212) that a patient should receive a cardiac valve procedure (e.g., TAVR, SAVR or another procedure) and/or the cardiac care pathway management module determines or infer that a patient should receive the procedure within a defined timeframe (e.g., six months, three months, one month, one week, etc.), the ordering component 220 can automatically order medical supplies (e.g., the recommended replacement valve and optionally other supplies) for the procedure to ensure the supplies are available and ready for performing the procedure. For example, the ordering component 220 can interface with one or more medical supply ordering systems 110 and order the one or more medical supplies required for the procedure, such as the particular replacement valve determined to be most appropriate for the patient and the procedure by the procedure assessment component 206. As a result of ordering the one or more medical supplies with the one or more medical supply ordering systems 110, the one or more medical supplies can be shipped/delivered to the physical location where the procedure will be performed on the patient at a time prior to performance of the procedure on the patient.

In some embodiments, the ordering component 220 can also facilitate automatically scheduling one or more medical appointments for patients evaluated by the system 100 based on the some or portions of the input data and/or the output data 128 satisfying one or more predefined appointment scheduling criterion. In this regard, the ordering component 220 can interface with one or more appointment scheduling systems 116 to identify available medical appointments with respective patients' designated care providers (e.g., primary care providers, specialty care providers, diagnostic procedure providers, etc.) and schedule one or more medical appointments for the patients with one or more of their designated care providers in accordance with a predefined cardiac care pathway protocol (e.g., defined in the cardiac care pathway domain knowledge systems/sources 120 and/or in another clinical data source/system of the other clinical data sources/systems). In this regard, the predefined cardiac care pathway protocol can define a structured protocol of the specific types of medical appointments to be scheduled for a patient based on the some or portions of the input data 126 satisfying a defined appointment criterion and/or some or portions of the output data 128 satisfying a defined appointment scheduling criterion. For example, the defined appointment scheduling criterion may instruct the ordering component 220 to schedule or order an imaging study (e.g., a heat/chest imaging study of a particular type such as MR, CT, XR, etc.) to be performed on a patient in response to a determination or inference by the condition assessment component 204 that the patient has a change in severity of their valvular heart condition. In another example, the defined appointment scheduling criterion may instruct the ordering component 220 to schedule an appointment with a patient's cardiologist in response to a determination by the procedure assessment component 206 and/or the recommendation component 208 that a TAVR or SAVR procedure is recommended for the patient within a defined timeframe.

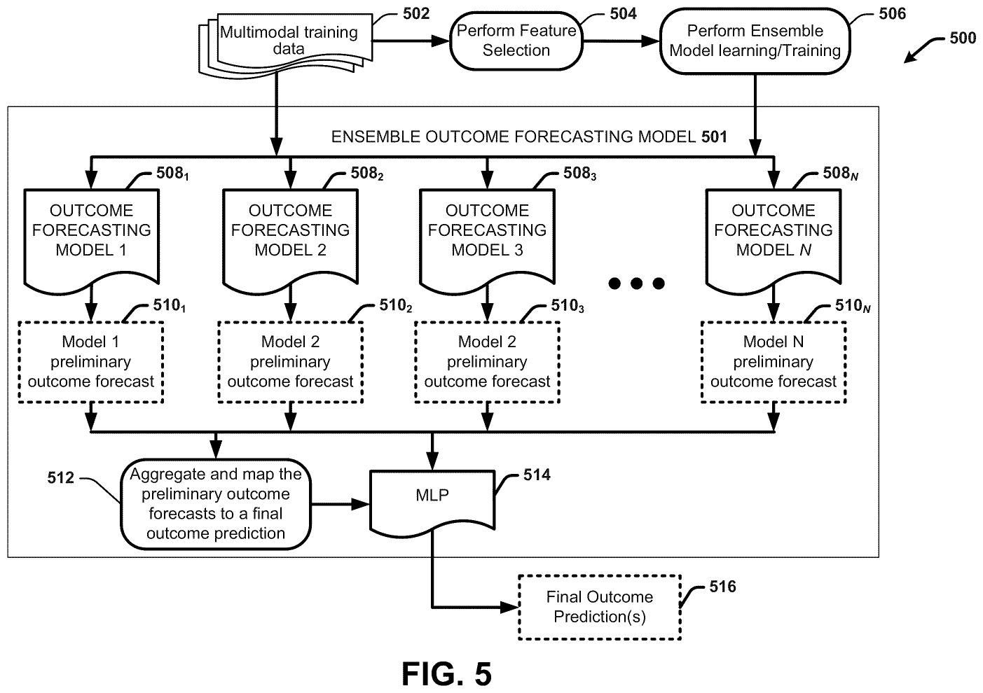

FIG. 5 illustrates a flow diagram of an example ensemble model training processes 500 for training one or more of the outcomes forecasting models 306 in accordance with one or more embodiments. In this regard, with reference to FIGS. 1-5, in some embodiments, one or more of the outcomes forecasting models 306 correspond to an ensemble outcome forecasting model 501. With these embodiments, the ensemble outcome forecasting model 501 can include a plurality of different sub-models corresponding to different types of machine learning models. These sub-models are represented in FIG. 5 as outcome forecasting models 508_{1-N}. The number of the different sub-models N employed and the respective types of machine learning models of the respective sub-models can vary. For example, in some implementations, the different types can include a Random Forest (RF) model type, an XGBoost model type, a Non-linear Kernel SVM model type, a neural network model type, a deep learning neural network model type, and other types of machine learning models.

In accordance with process 500, each of the different outcome forecasting models 508_{1-N} are individually trained (e.g., via the training component 210) to predict one or more patient outcomes using multimodal training data 502 (e.g., included in training data 228) for a plurality of different AS patients that received a cardiac valve procedure in the past (e.g., SAVR and/or TAVR) and for which their outcomes have been recorded or documented.

In some embodiments, each of the different sub-models can respectively be independently trained to predict expected LOS and/or probability of readmission after receiving a valvular heart procedure, such as TAVR and/or SAVR. In some implementations, the predicted LOS can comprise a value that represents the predicted LOS of a patient in the hospital following reception of the cardiac valve procedure. In other embodiments, the predicted LOS can include a binary value that indicates whether the expected LOS exceeds a threshold LOS considered normal or acceptable. In other words, the LOS value output by the respective models can indicate whether the patient is expected to have a longer than usual LOS. In some embodiments, the probability of readmission can also include a binary value that indicates whether the patient is expected to be readmitted (or not) within a defined timeframe following discharge.

In some embodiments, each of the different sub-models (e.g., outcome forecasting models 508_{1-N}) can respectively be trained to predict both outcomes using the same multimodal training data 502 input. In other embodiments, a first subset of the different sub-models (e.g., the first subset including one or more models) can be trained to predict the LOS valve and a second subset of the different sub-models (e.g., the second subset including one or more models) can be trained to predict the readmission risk valve.

In accordance with process 500, at 504, the training component 210 can perform feature selection to select and index the relevant input data features for processing by the respective sub-models. In this regard, the multimodal training data 502 can include a plurality of different features for different AS patients before undergoing aortic valve replacement (AVR) procedures, including surgical aortic valve replacement (SAVR) and transcatheter aortic valve replacement (TAVR). In some embodiments, at 504, the training component 210 can generate an indexed set of input features for the respective models. For example, the training component 210 can formulate the different features as tabular values, with each row representing a patient and each column representing a specific patient feature, including demographics (e.g., age, gender, BMI), laboratories, comorbidities, symptoms, medications, diagnosis, AS severity, replacement valve utilized, and various other features described herein. At 506, the training component 210 can perform ensemble model learning training using available information for the respective patients regarding known outcomes observed for the respective patients (e.g., LOS, readmission rate, complications observers, etc.) can be used as the ground truth information for model training in accordance with a supervised machine learning training process.

In various embodiments, the ensemble models learning/training process can involve separately training each of the different outcome models 508_{1-N} to predict preliminary outcome forecast data models 510_{1-N}. At 512, the training component can further aggregate and map the preliminary outcome forecasts generated by the respective sub-models to a final outcome prediction 516. In various embodiments, this is accomplished using a two-layer MLP 514, which is trained by the training component 210 by mapping the different outputs from the different sub-models to the patient outcomes.

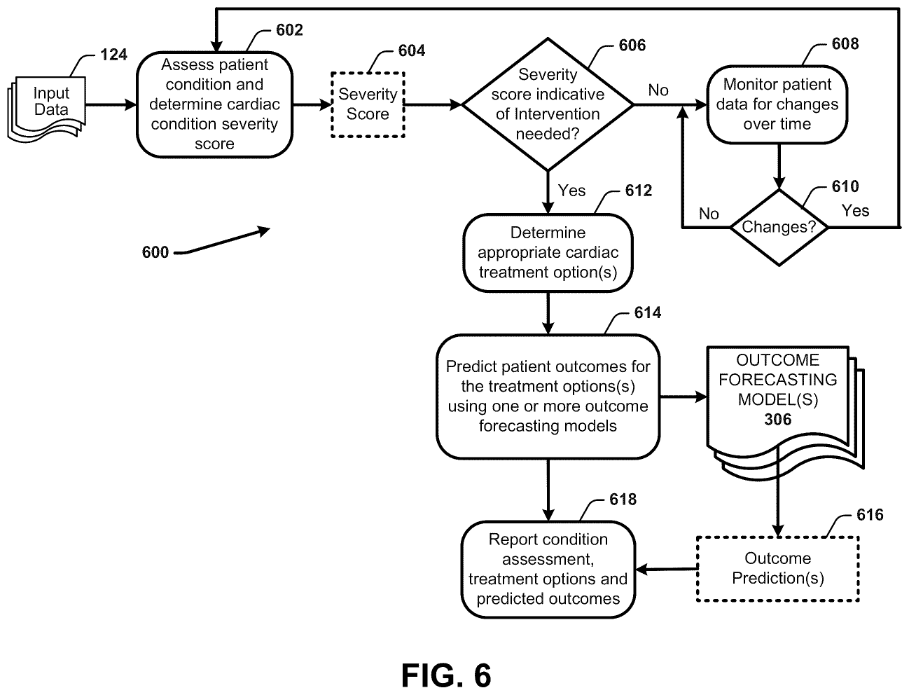

FIG. 6 presents a flow diagram of an example process 600 for early detection and management of patients with valvular heart disease using machine learning analytics in accordance with one or more embodiments of the disclosed subject matter. Repetitive description of like elements employed in respective embodiments is omitted for sake of brevity.

In accordance with process 600, in association with collection of the input data 126 for one or more patients, for each patient represented in the input data, at 602 the condition assessment component 204 can assess the patient condition and determine a cardiac condition severity score 604 that provides a measure of severity of their cardiac disease. At 606, the condition assessment component 204 can determine whether the severity score indicates an intervention is needed for the patient based on whether the severity score exceeds a threshold severity score. If at 606 the condition assessment component determines that the severity score is not indicative of the patient needing intervention (e.g., via SAVR and/or TAVR), process 600 can proceed to 608, wherein the data collection component monitors the patient data for changes over time. If changes are detected at 610, then process 600 returns to 602 and the condition assessment component 204 reassesses the patient's condition and severity score.

If at 606 the condition assessment component 204 determines that the severity score indicates intervention is needed for the patient, process 600 then proceeds to 612, wherein the procedure assessment component 206 determines one or more appropriate cardiac treatment options for the patient (e.g., TAVR and/or SAVR). At 614, the outcomes forecasting component 208 then predicts one or more patient outcomes 616 for the respective treatment options using one or more outcomes forecasting modes 306. In some embodiments, the one or more outcome forecasting models 306 can include or correspond to ensemble outcome forecasting model 501. At 610, the reporting component can further report the results of the condition assessment, the treatment options and the predicted outcomes using a suitable electronic reporting mechanism.

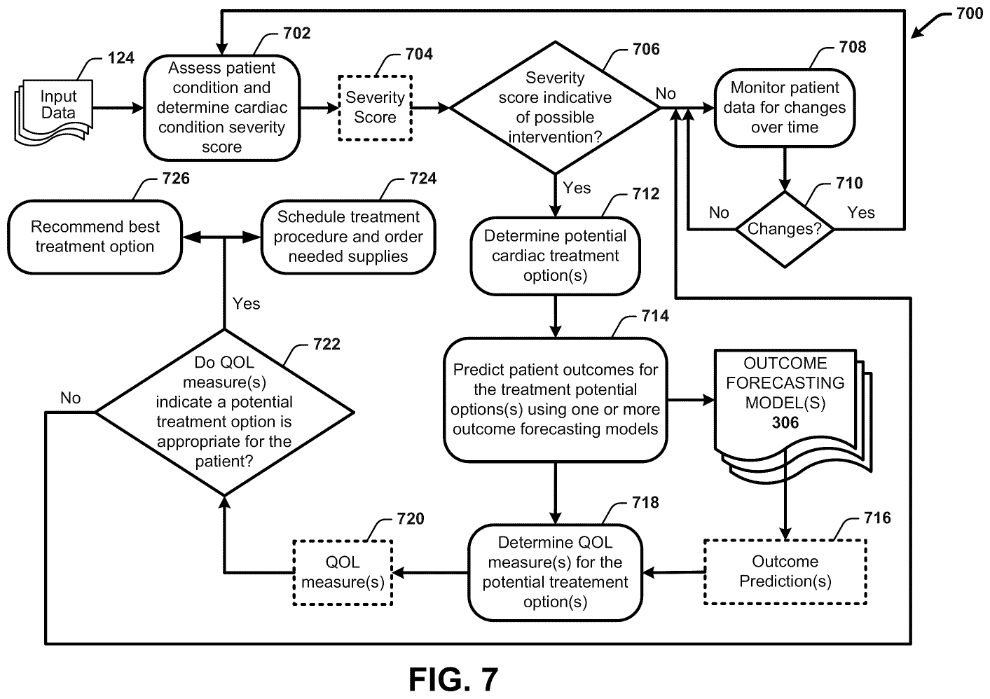

FIG. 7 presents a flow diagram of another example process 700 for early detection and management of patients with valvular heart disease using machine learning analytics in accordance with one or more embodiments of the disclosed subject matter. Repetitive description of like elements employed in respective embodiments is omitted for sake of brevity.

In accordance with process 700, in association with collection of the input data 126 for one or more patients, for each patient represented in the input data, at 702 the condition assessment component 204 can assess the patient condition and determine a cardiac condition severity score 704 that provides a measure of severity of their cardiac disease. At 706, the condition assessment component 204 can determine whether the severity score indicates an intervention is needed for the patient based on whether the severity score exceeds a threshold severity score. If at 706 the condition assessment component determines that the severity score is not indicative of the patient needing intervention (e.g., via SAVR and/or TAVR), process 700 can proceed to 708, wherein the data collection component 202 monitors the patient data for changes over time. If changes are detected at 710, then process 700 returns to 702 and the condition assessment component 704 reassesses the patient's condition and severity score.

If at 706 the condition assessment component 204 determines that the severity score indicates intervention is needed for the patient, process 700 then proceeds to 712, wherein the procedure assessment component 206 determines one or more potential cardiac treatment options for the patient (e.g., TAVR and/or SAVR). At 714, the outcomes forecasting component 208 then predicts one or more patient outcomes 716 for the respective treatment options using one or more outcomes forecasting modes 306. In some embodiments, the one or more outcome forecasting models 306 can include or correspond to ensemble outcome forecasting model 501. In some embodiments, the outcome predictions 716 can include a first measure indicating whether the patient is expected to experience a longer than usual LOS after receiving the procedure and a second measure indicative of whether the patient is expected to be readmitted post discharge after receiving the procedure withing a defined time frame (e.g., six months or another time frame).

At 710, the outcomes forecasting component 208 can further determine one more QOL measures 720 for the potential treatment options. At 722, the recommendation component 212 can determine whether the QOL measures indicate a potential treatment option is appropriate for the patient or not based on whether the QOL measure satisfy a defined criterion (e.g., exceed a minimum QOL improvement threshold or the like). If the recommendation component determines at 722 that proceeding with any of the potential treatment options (e.g., TAVR and/or SAVR) is inappropriate for the patient, then process 700 proceeds back to 708, wherein the data collection component monitors the patient data for changes over time. However, if at 722 the recommendation component determines that one or more of the treatment options are appropriate for the patient, then the recommendation component can recommend the best treatment option for the patient at 726 and/or the ordering component 220 can schedule the treatment procedure and order one or more supplies needed for the procedure.

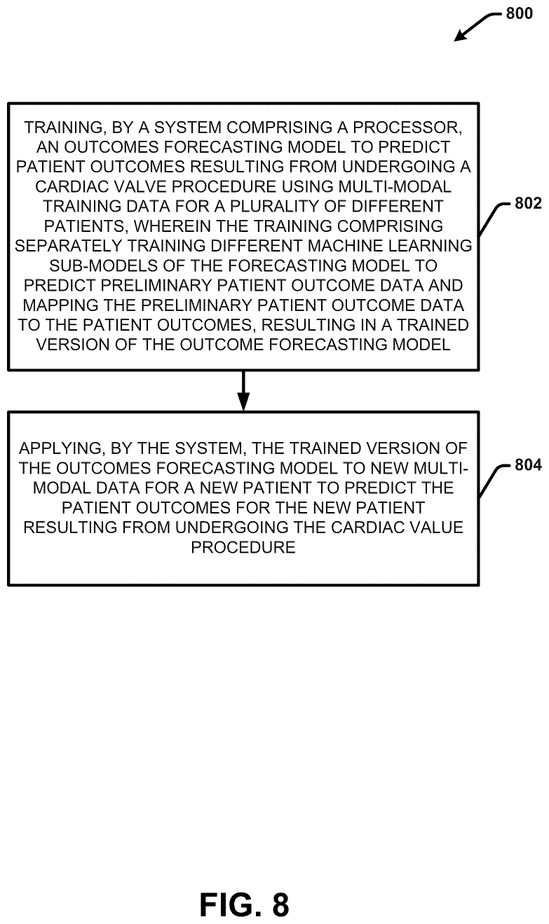

FIG. 8 presents an example computer-implemented process 800 that facilitates early detection and management of patients with valvular heart disease in accordance with one or more embodiments of the disclosed subject matter. Repetitive description of like elements employed in respective embodiments is omitted for sake of brevity.

In accordance with process 800, at 802 a system operatively coupled to a processor (e.g., system 100 or the like), trains an outcomes forecasting model (e.g., ensemble outcome forecasting model 501) o predict patient outcomes resulting from undergoing a cardiac valve procedure using multi-modal training data (e.g., multimodal training data 502) for a plurality of different patients, wherein the training comprising separately training different machine learning sub-models (e.g., outcome forecasting models 508_{1-N}) of the forecasting model to predict preliminary patient outcome data (e.g., preliminary outcome forecast data 510_{1-N}) and mapping the preliminary patient outcome data to the patient outcomes (e.g., using a MLP 514), resulting in a trained version of the outcome forecasting model. At 804, method 800 further comprises applying, by the system (e.g., via the outcomes forecasting component 208), the trained version of the outcomes forecasting model to new multi-modal data for a new patient (e.g., included in input data 126) to predict the patient outcomes for the new patient resulting from undergoing the cardiac value procedure.

In some embodiments, the patient outcomes include a LOS forecast (e.g., expected LOS and/or a binary value indicative of whether the patient is expected to experience LOS exceeding a normal LOS value) and a readmission risk forecast (e.g., one or more measure indicative of whether the patient is expected to be readmitted within a defined timeframe). In some embodiments, method 800 can further include performing, by the system, an assessment of a valvular heart disease of the new patient based on the analysis of new multi-modal patient data for the new patient, the new muti-modal data representing condition parameters for the patient at different points in time up to a current point in time; determining, by the system, a measure of severity of the valvular heart condition for the new patient based on the assessment; and determining, by the system, whether the cardiac valve procedure is appropriate for the new patient based on whether the severity measure exceeds a threshold (e.g., using condition assessment component 204). In some embodiments, method 800 can further comprise receiving, by the system, respective components of the new multi-modal patient data via a network (e.g., one or more networks 106) from one or more electronic healthcare information sources (e.g., electronic healthcare information sources 124) as the respective components are received at the one or more electronic healthcare information sources; and performing, by the system, the condition assessment repeatedly based on reception of new components of the respective components.

In various embodiments, the cardiac valve procedure comprises different types of procedures (e.g., SAVR, TAVR and other types of procedures). With these embodiments, method 800 can further comprise, based on the severity measure exceeding the threshold, determining, by the system, a preferred procedure type of the different types of procedures for performing on the new patient based on the new multi-modal patient data and the severity measure, and wherein the patient outcomes predicted by the outcomes forecasting model for the new patient are a function of the preferred procedure type. In some implementations of these embodiments the surgical cardiac value procedure comprises an aortic valve replacement procedure and wherein the different types of procedures comprise SAVR and TAVR and method 800 can further comprise determining, by the system, one or more replacement valves to use for the cardiac valve procedure. In various embodiments, the system can determine (e.g., using procedure assessment component 206) the preferred procedure type and the one or more replacement valves based on a plurality of criteria selected from the group consisting of: annulus size, distance of coronary artery from AV annulus, severe aortic valve calcification, aneurysm of ascending aorta, position and tortuosity of ascending aorta, bicuspid aortic valve, access route tortuosity, small vessel diameter, left ventricular function, coronary artery disease, and ECG conduction abnormalities.

### EXAMPLE OPERATING ENVIRONMENTS

One or more embodiments can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. In this regard, in one or more embodiments, a compute readable storage medium, as used herein, can include or correspond to a non-transitory machine-readable storage medium.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 9, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 9, an example environment 900 for implementing various aspects of the claimed subject matter includes a computer 902. The computer 902 includes a processing unit 904, a system memory 906, a codec 935, and a system bus 908. The system bus 908 couples system components including, but not limited to, the system memory 906 to the processing unit 904. The processing unit 904 can be any of various available processors. Dual microprocessors and other multiprocessor architectures also can be employed as the processing unit 904.

The system bus 908 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 906 includes volatile memory 910 and non-volatile memory 912, which can employ one or more of the disclosed memory architectures, in various embodiments. The basic input/output system (BIOS), containing the basic routines to transfer information between elements within the computer 902, such as during start-up, is stored in non-volatile memory 912. In addition, according to present innovations, codec 935 can include at least one of an encoder or decoder, wherein the at least one of an encoder or decoder can consist of hardware, software, or a combination of hardware and software. Although, codec 935 is depicted as a separate component, codec 935 can be contained within non-volatile memory 912. By way of illustration, and not limitation, non-volatile memory 912 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 912 can employ one or more of the disclosed memory devices, in at least some embodiments. Moreover, non-volatile memory 912 can be computer memory (e.g., physically integrated with computer 902 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 910 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 902 can also include removable/non-removable, volatile/non-volatile computer storage medium. FIG. 9 illustrates, for example, disk storage 914. Disk storage 914 includes, but is not limited to, devices like a magnetic disk drive, solid state disk (SSD), flash memory card, or memory stick. In addition, disk storage 914 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 914 to the system bus 908, a removable or non-removable interface is typically used, such as interface 916. It is appreciated that disk storage 914 can store information related to a user. Such information might be stored at or provided to a server or to an application running on a user device. In one embodiment, the user can be notified (e.g., by way of output device(s) 936) of the types of information that are stored to disk storage 914 or transmitted to the server or application. The user can be provided the opportunity to opt-in or opt-out of having such information collected or shared with the server or application (e.g., by way of input from input device(s) 928).

It is to be appreciated that FIG. 9 describes software that acts as an intermediary between users and the basic computer resources described in the suitable operating environment 900. Such software includes an operating system 910. Operating system 910, which can be stored on disk storage 914, acts to control and allocate resources of the computer 902. Applications 920 take advantage of the management of resources by operating system 910 through program modules 924, and program data 926, such as the boot/shutdown transaction table and the like, stored either in system memory 906 or on disk storage 914. It is to be appreciated that the claimed subject matter can be implemented with various operating systems or combinations of operating systems.

A user enters commands or information into the computer 902 through input device(s) 928. Input devices 928 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 904 through the system bus 908 via interface port(s) 930. Interface port(s) 930 include, for example, a serial port, a parallel port, a game port, and a universal serial bus (USB). Output device(s) 936 use some of the same type of ports as input device(s) 928. Thus, for example, a USB port can be used to provide input to computer 902 and to output information from computer 902 to an output device 936. Output adapter 934 is provided to illustrate that there are some output devices 936 like monitors, speakers, and printers, among other output devices 936, which require special adapters. The output adapters 934 include, by way of illustration and not limitation, video and sound cards that provide a means of connection between the output device 936 and the system bus 908. It should be noted that other devices or systems of devices provide both input and output capabilities such as remote computer(s) 938.

Computer 902 can operate in a networked environment using logical connections to one or more remote computers, such as remote computer(s) 938. The remote computer(s) 938 can be a personal computer, a server, a router, a network PC, a workstation, a microprocessor based appliance, a peer device, a smart phone, a tablet, or other network node, and typically includes many of the elements described relative to computer 902. For purposes of brevity, only a memory storage device 940 is illustrated with remote computer(s) 938. Remote computer(s) 938 is logically connected to computer 902 through a network interface 942 and then connected via communication connection(s) 944. Network interface 942 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 944 refers to the hardware/software employed to connect the network interface 942 to the bus 908. While communication connection 944 is shown for illustrative clarity inside computer 902, it can also be external to computer 902. The hardware/software necessary for connection to the network interface 942 includes, for exemplary purposes only, internal and external technologies such as, modems including regular telephone grade modems, cable modems and DSL modems, ISDN adapters, and wired and wireless Ethernet cards, hubs, and routers.

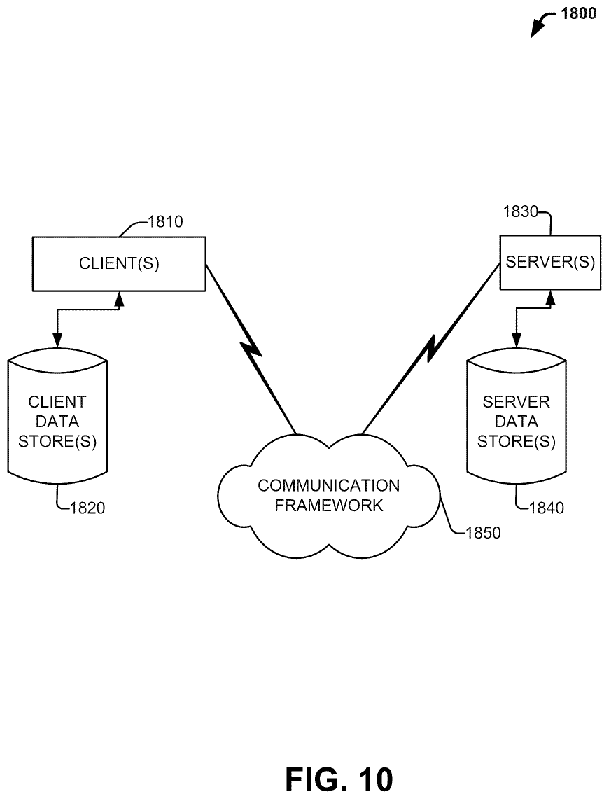

FIG. 10 is a schematic block diagram of a sample-computing environment 1000 with which the subject matter of this disclosure can interact. The system 1000 includes one or more client(s) 1010. The client(s) 1010 can be hardware and/or software (e.g., threads, processes, computing devices). The system 1000 also includes one or more server(s) 1030. Thus, system 1000 can correspond to a two-tier client server model or a multi-tier model (e.g., client, middle tier server, data server), amongst other models. The server(s) 1030 can also be hardware and/or software (e.g., threads, processes, computing devices). The servers 1030 can house threads to perform transformations by employing this disclosure, for example. One possible communication between a client 1010 and a server 1030 may be in the form of a data packet transmitted between two or more computer processes.

The system 1000 includes a communication framework 1050 that can be employed to facilitate communications between the client(s) 1010 and the server(s) 1030. The client(s) 1010 are operatively connected to one or more client data store(s) 1020 that can be employed to store information local to the client(s) 1010. Similarly, the server(s) 1030 are operatively connected to one or more server data store(s) 1040 that can be employed to store information local to the servers 1030.

It is to be noted that aspects or features of this disclosure can be exploited in substantially any wireless telecommunication or radio technology, e.g., Wi-Fi; Bluetooth; Worldwide Interoperability for Microwave Access (WiMAX); Enhanced General Packet Radio Service (Enhanced GPRS); Third Generation Partnership Project (3GPP) Long Term Evolution (LTE); Third Generation Partnership Project 2 (3GPP2) Ultra Mobile Broadband (UMB); 3GPP Universal Mobile Telecommunication System (UMTS); High Speed Packet Access (HSPA); High Speed Downlink Packet Access (HSDPA); High Speed Uplink Packet Access (HSUPA); GSM (Global System for Mobile Communications) EDGE (Enhanced Data Rates for GSM Evolution) Radio Access Network (GERAN); UMTS Terrestrial Radio Access Network (UTRAN); LTE Advanced (LTE-A); etc. Additionally, some or all of the aspects described herein can be exploited in legacy telecommunication technologies, e.g., GSM. In addition, mobile as well non-mobile networks (e.g., the Internet, data service network such as internet protocol television (IPTV), etc.) can exploit aspects or features described herein.

While the subject matter has been described above in the general context of computer-executable instructions of a computer program that runs on a computer and/or computers, those skilled in the art will recognize that this disclosure also can or may be implemented in combination with other program modules. Generally, program modules include routines, programs, components, data structures, etc. that perform particular tasks and/or implement particular abstract data types. Moreover, those skilled in the art will appreciate that the inventive methods may be practiced with other computer system configurations, including single-processor or multiprocessor computer systems, mini-computing devices, mainframe computers, as well as personal computers, hand-held computing devices (e.g., PDA, phone), microprocessor-based or programmable consumer or industrial electronics, and the like. The illustrated aspects may also be practiced in distributed computing environments where tasks are performed by remote processing devices that are linked through a communications network. However, some, if not all aspects of this disclosure can be practiced on stand-alone computers. In a distributed computing environment, program modules may be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

Various aspects or features described herein can be implemented as a method, apparatus, system, or article of manufacture using standard programming or engineering techniques. In addition, various aspects or features disclosed in this disclosure can be realized through program modules that implement at least one or more of the methods disclosed herein, the program modules being stored in a memory and executed by at least a processor. Other combinations of hardware and software or hardware and firmware can enable or implement aspects described herein, including a disclosed method(s). The term “article of manufacture” as used herein can encompass a computer program accessible from any computer-readable device, carrier, or storage media. For example, computer readable storage media can include but are not limited to magnetic storage devices (e.g., hard disk, floppy disk, magnetic strips . . . ), optical discs (e.g., compact disc (CD), digital versatile disc (DVD), blu-ray disc (BD) . . . ), smart cards, and flash memory devices (e.g., card, stick, key drive . . . ), or the like.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or methods herein are intended to include, without being limited to including, these and any other suitable types of memory.

It is to be appreciated and understood that components, as described with regard to a particular system or method, can include the same or similar functionality as respective components (e.g., respectively named components or similarly named components) as described with regard to other systems or methods disclosed herein.

What has been described above includes examples of systems and methods that provide advantages of this disclosure. It is, of course, not possible to describe every conceivable combination of components or methods for purposes of describing this disclosure, but one of ordinary skill in the art may recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

What is claimed is:

1. A system, comprising:
a memory that stores computer-executable components; and
a processor that executes the computer-executable components stored in the memory, wherein the computer-executable components comprise:
a training component that trains an outcomes forecasting model to predict patient outcomes resulting from undergoing a cardiac valve procedure using multi-modal training data for a plurality of different patients, wherein the training comprising separately training different machine learning sub-models of the forecasting model to predict preliminary patient outcome data and mapping the preliminary patient outcome data to the patient outcomes, resulting in a trained version of the outcome forecasting model; and
an outcomes forecasting component that applies the trained version of the outcomes forecasting model to new multi-modal data for a new patient to predict the patient outcomes for the new patient resulting from undergoing the cardiac value procedure.

2. The system of claim 1, wherein the patient outcomes include a length of stay and a readmission risk.

3. The system of claim 1, wherein the patient outcomes include at least one of: a change to one or more defined physiological parameters, potential adverse reactions, and a change to a severity measure representative of a measure of severity of a cardiac condition of the new patient.

4. The system of claim 1, wherein the computer-executable components further comprise:
a condition assessment component that performs an assessment of a valvular heart disease of the new patient based on the analysis of the new multi-modal data for the new patient and determines a measure of severity of the valvular heart disease relative to the new patient based on the assessment, the new muti-modal data representing physiological condition parameters for the new patient at different points in time up to a current point in time, wherein the condition assessment component determines whether the cardiac valve procedure is appropriate for the new patient based on whether the measure of severity exceeds a threshold.

5. The system of claim 4, wherein the outcomes forecasting component applies the trained version of the outcomes forecasting model to the new multi-modal data for the new patient based on the measure of severity exceeding the threshold.

6. The system of claim 4, wherein the computer-executable components further comprise:
a data collection component that receives respective components of the new multi-modal data via a network from one or more electronic healthcare information sources as the respective components are received at the one or more electronic healthcare information sources, and wherein the condition assessment component performs the assessment repeatedly based on reception of new components of the respective components.

7. The system of claim 4, wherein the cardiac valve procedure comprises different types of procedures and wherein the computer-executable components further comprise:
a procedure assessment component that determines, a preferred procedure type of the different types of procedures for performing on the new patient based on the new multi-modal data and the severity measure, and wherein the patient outcomes predicted by the outcomes forecasting model for the new patient are a function of the preferred procedure type.

8. The system of claim 7, wherein the cardiac value procedure comprises an aortic valve replacement procedure and wherein the different types of procedures comprise a surgical aortic valve replacement (SAVR) procedure type and a transcatheter aortic valve replacement (TAVR) procedure type, and wherein the procedure assessment component further determines one or more replacement valves to use for the cardiac valve procedure.

9. The system of claim 8, wherein the procedure assessment component determines the preferred procedure type and the one or more replacement valves based on a plurality of criteria selected from the group consisting of: annulus size, distance of coronary artery ostia from AV annulus, severe aortic valve calcification, aneurysm of ascending aorta, position and tortuosity of ascending aorta, bicuspid aortic valve, access route tortuosity, small vessel diameter, left ventricular function, coronary artery disease, and ECG conduction.

10. The system of claim 4, wherein the computer-executable components further comprise:
a recommendation component that determines a recommendation regarding whether the new patient should proceed or not proceed with the cardiac valve procedure based on the patient outcomes predicted for the new patient and the measure of severity; and
a reporting component that generates report data identifying the recommendation, the severity measure and the patient outcomes predicted for the new patient and provides the report data to one or more entities using an electronic reporting mechanism.

11. The system of claim 10, wherein the recommendation component determines the recommendation based on a value output by a machine learning model generated based on application of the machine learning model to input data comprising the new multi-modal data, the measure of severity and the patient outcomes predicted for the new patient, the value indicative of a degree to which proceeding with the cardiac valve procedure will improve a quality of life of the patient, wherein the training component trains the machine learning model using a training dataset comprising historical data for past patients including patients that received and did not receive the cardiac valve procedure.

12. The system of claim 10, wherein the computer-executable components further comprise:
an ordering component that determines one or more medical supplies needed for the proceeding with the cardiac valve procedure based on the recommendation indicating the new patient should proceed with the cardiac valve procedure and orders the one or more medical supplies using an electronic medical supply ordering system.

13. The system of claim 1, wherein the multi-modal training data and the multi-modal data for the new patient comprises electronic medical record data, echocardiogram study findings data, and electrocardiogram study finding data.

14. A method, comprising:
training, by a system comprising a processor, an outcomes forecasting model to predict patient outcomes resulting from undergoing a cardiac valve procedure using multi-modal training data for a plurality of different patients, wherein the training comprising separately training different machine learning sub-models of the forecasting model to predict preliminary patient outcome data and mapping the preliminary patient outcome data to the patient outcomes, resulting in a trained version of the outcome forecasting model; and
applying, by the system, the trained version of the outcomes forecasting model to new multi-modal data for a new patient to predict the patient outcomes for the new patient resulting from undergoing the cardiac value procedure.

15. The method of claim 14, wherein the patient outcomes include at least one of: a length of stay, a readmission risk, a change to one or more defined physiological parameters, potential adverse reactions, and a change to a severity measure representative of a measure of severity of a cardiac condition of the new patient.

16. The method of claim 14, further comprising
performing, by the system, an assessment of a valvular heart disease of the new patient based on the analysis of the new multi-modal data for the new patient, the new muti-modal data representing condition parameters for the patient at different points in time up to a current point in time;
determining, by the system, a measure of severity of the valvular heart condition for the new patient based on the assessment; and
determining, by the system, whether the cardiac valve procedure is appropriate for the new patient based on whether the measure of severity exceeds a threshold.

17. The method of claim 16, further comprising:
receiving, by the system, respective components of the new multi-modal data via a network from one or more electronic healthcare information sources as the respective components are received at the one or more electronic healthcare information sources; and
performing, by the system, the assessment repeatedly based on reception of new components of the respective components.

18. The method of claim 16, wherein the cardiac valve procedure comprises different types of procedures and wherein the method further comprises, based on the measure of severity exceeding the threshold:
determining, by the system, a preferred procedure type of the different types of procedures for performing on the new patient based on the new multi-modal data and the measure of severity, and wherein the patient outcomes predicted by the outcomes forecasting model for the new patient are a function of the preferred procedure type.

19. The method of claim 18, wherein the surgical cardiac value procedure comprises an aortic valve replacement procedure and wherein the different types of procedures comprise a surgical aortic valve replacement (SAVR) procedure type and a transcatheter aortic valve replacement (TAVR) procedure type, and wherein the method further comprises:
determining, by the system, one or more replacement valves to use for the cardiac valve procedure, and wherein determining the preferred procedure type and the one or more replacement valves comprises determining the preferred procedure type and the one or more replacement valves based on a plurality of criteria selected from the group consisting of: annulus size, distance of coronary artery ostia from AV annulus, severe aortic valve calcification, aneurysm of ascending aorta, position and tortuosity of ascending aorta, bicuspid aortic valve, access route tortuosity, small vessel diameter, left ventricular function, coronary artery disease, and ECG conduction abnormalities.

20. A non-transitory machine-readable storage medium, comprising executable instructions that, when executed by a processor, facilitate performance of operations, comprising:
training an outcomes forecasting model to predict patient outcomes resulting from undergoing a cardiac valve procedure using multi-modal training data for a plurality of different patients, wherein the training comprising separately training different machine learning sub-models of the forecasting model to predict preliminary patient outcome data and mapping the preliminary patient outcome data to the patient outcomes, resulting in a trained version of the outcome forecasting model; and
applying the trained version of the outcomes forecasting model to new multi-modal data for a new patient to predict the patient outcomes for the new patient resulting from undergoing the cardiac value procedure.
